(19) United States
(12) Patent Application Publication
(10) Pub. No.: US 2006/0211005 A1
(43) Pub. Date: Sep. 21, 2006

(54) Method of measuring pyrophosphate and method of detecting primer extension reaction, and device for performing the same

(75) Inventors: Hidenobu Yaku, Osaka (JP); Masayoshi Maeshima, Nagoya-shi (JP); Yoichi Nakanishi, Nagoya-shi (JP); Megumi Hirono, Nukata-gun (JP); Tetsuo Yukimasa, Nara-shi (JP); Hiroaki Oka, Osaka (JP)

Correspondence Address: McDERMOTT WILL & EMERY LLP, 600 13th Street, N.W., Washington, DC, 20005-3096, US

(73) Assignee: MATSUSHITA ELECTRIC INDUSTRIAL CO., LTD., Osaka (JP)

(21) Appl. No.: 11/180,881

(22) Filed: Jul. 14, 2005

Related U.S. Application Data
(63) Continuation of application No. PCT/JP05/05522, filed on Mar. 25, 2005.

(30) Foreign Application Priority Data
Mar. 29, 2004 (JP) 2004-095154

(51) Int. Cl. C12Q 1/68; C12Q 1/34

(52) U.S. Cl. 435/6; 435/18

(57) ABSTRACT

An object of the present invention is to provide a method of quantitative determination of pyrophosphate (PPi) and a method of detecting a primer extension reaction that require fewer kinds of enzymes and that do not necessitate strict temperature regulation, and a kit and a device for performing these methods.

In the method of measuring PPi of the present invention, a PPi sample having an unknown concentration is allowed to act on mycobacterial H⁺-pyrophosphatase (H⁺-PPase) intrinsically included in membrane, thereby measuring the PPi concentration in the sample by analyzing thus resulting H⁺transport. Furthermore, in the method of detecting an extension reaction of a primer of the present invention, an unknown nucleic acid sample to which examination of occurrence of the primer extension reaction is intended is allowed to act on mycobacterial H⁺-PPase intrinsically included in a membrane, thereby determining occurrence of the primer extension reaction on the unknown nucleic acid sample by analyzing thus resulting H⁺transport.

This is a continuation application under U.S.C 111(a) of pending prior International application No. PCT/JP2005/005522, filed on Mar. 25, 2005.

### BACKGROUND OF THE INVENTION

1. Field of the Invention

The present invention relates to a method of measuring pyrophosphate and a method of detecting a base sequence of a particular nucleic acid or a particular base type, and a device for measuring pyrophosphate and a device for a primer extension reaction for performing the methods.

2. Description of the Related Art

Pyrophosphate (hereinafter, referred to as “PPi”) has been known to be prominently involved in enzyme reactions in cells. For example, in the course of protein synthesis, PPi is produced in a reaction to form amino acyl tRNA from an amino acid via aminoacyl adenylate. Further, in the course of starch synthesis, for example, found in plants and the like, PPi is produced when ADP-glucose is produced by a reaction of glucose-1-phosphate with ATP. In addition thereto, PPi has been known to be involved in a variety of enzyme reactions. Therefore, techniques to quantitatively detect PPi are important in analyses of cellular states, or enzyme reactions as described above, and the like.

A chemical method of Grindley et al., (G. B. Grindley and C. A. Nichel, Anal. Biochem., Vol. 33, p. 114 (1970)) is known as a conventional method of measuring PPi. However, because this method uses concentrated sulfuric acid, it is not preferred in light of safety.

Japanese Patent Provisional Publication. S61-12300 discloses three kinds of methods of the measurement of PPi in which an enzyme is used without use of hazardous chemicals such as concentrated sulfuric acid.

In the first method, pyruvate orthophosphate dikinase is allowed to act on PPi in the presence of phosphoenolpyruvate and adenosine monophosphate. Because this reaction produces pyruvic acid, the amount of PPi can be calculated through the determination of the amount of pyruvic acid. For reference, two kinds of methods have been proposed as the method of the determining the amount of pyruvic acid. In one method, when a catalytic action of lactate dehydrogenase is utilized to reduce pyruvic acid with NADH, decrease in NADH is calorimetrically determined. In another method, pyruvate oxidase is allowed to act on the produced pyruvic acid, and colorimetric determination is carried out through introducing thus produced hydrogen peroxide to a dye.

In the second method, PPi is allowed to act on glycerol-3-phosphate cytidyl transferase in the presence of cytidine diphosphoglycerol. This reaction results in production of glycerol triphosphate. Therefore, amount of PPi can be calculated by determining the amount of production of glycerol triphosphate. Two kinds of methods have been proposed as the method of the determining the amount of glycerol triphosphate. In one method, when a catalytic action of glycerol-3-phosphate dehydrogenase is utilized to oxidize glycerol triphosphate with NAD(P), increase in NAD(P)H is calorimetrically determined. In another method, glycerol-3-phosphateoxidase is allowed to act on the produced glycerol triphosphate, and calorimetric determination is carried out through introducing thus produced hydrogen peroxide to a dye.

In the third method, ribitol-5-phosphate cytidyl transferase is allowed to act on PPi in the presence of cytidine diphosphate ribitol. Because this reaction produces D-ribitol-5-phosphate, the amount of PPi can be calculated through the determination of the amount of production. As the method of the determination of D-ribitol-5-phosphate, a method in which ribitol-5-phosphate dehydrogenase is allowed to act in the presence of NAD (or NADP), and increase in NADH (or NADPH) is calorimetrically determined.

Also, in addition to the methods described above, a method in which PPi is converted to ATP followed by utilization of a luciferase reaction has been known.

Furthermore, the techniques of the measurement of PPi as described above can be applied not only to mere measurement of PPi, but also, for example, to detection of a base sequence of a particular nucleic acid in which a method of nucleic acid amplification is utilized, which is typified by PCR method. In the method, although the presence/absence of a base sequence of a particular target nucleic acid in a sample can be decided according to whether or not an extension reaction was carried out from a primer that specifically binds to the target nucleic acid sequence, production of PPi has been known as a byproduct in the primer extension reaction.

Accordingly, because detection of PPi accompanied by a primer extension reaction (nucleic acid amplification reaction) directly leads to detection of a base sequence of a target nucleic acid, measurement of PPi by combined use of the primer extension reaction and any one of the aforementioned techniques of measuring PPi permits detection of the base sequence of the target nucleic acid. Such a technique can be applied to, for example, inspections on contamination of foods with bacteria and viruses, or inspections on infection of human body with bacteria and viruses.

Moreover, the technique of measuring PPi is also applicable to discriminate a particular base type within a nucleic acid base sequence. More specifically, it has been known that, for example, mutation of a particular single base within a certain gene causes a serious disease, or that gene polymorphism resulting from one base alteration, which is referred to as SNP, affects constitution of each individual. Thus, techniques to discriminate a base type of a particular single base have been particularly emphasized in recent years. As a representative technique among those, a method in which a primer extension reaction is utilized has been known.

This method specifies a base type through analyzing the presence/absence or difference in efficiency of a primer extension reaction which is dependent on the base type of a target base. Also in this method, the intended analysis can be accomplished by measuring the amount of PPi production accompanied by the reaction, similarly to the method to detect a nucleic acid base sequence as described above.

On one hand, H⁺-pyrophosphatase (hereinafter, referred to as “H⁺-PPase”) is an energy converting enzyme which converts energy released in a hydrolyzing step of a high energy phosphate bond of PPi into active transport of H⁺ via a membrane. Originally, H⁺-PPase was detected on its enzyme function in a photosynthetic bacterium (Rhodospilium rubrum), and with the progress of genome project in recent years, it has been elucidated to distribute in an unexpectedly broad spectrum in animate nature.

Accordingly, H⁺-PPase has been proven to exist in entire plant kingdom including higher plants and green algae, as well as cell membrane of a type of bacteria such as photosynthetic bacteria and archaebacteria, and membrane of intracellular acidic granule carried by parasitic protist such as Trypanosoma cruzi and malaria protozoan and the like. Among these, comparably well studied ones are H⁺-PPase found in plants, which are speculated to be a prerequisite enzyme for plants although there still left unexplained matters. However, there is no doubt about its importance any longer. More specifically, the explanation will be made as in the followings.

H⁺-PPase that is intrinsically present in plant tonoplast membrane conducts elimination of cytoplasmic PPi through hydrolysis to promote synthesis reactions of macromolecules in vivo. Furthermore, H⁺-PPase contributes in maintenance of cytoplasmic pH, acidification of vacuole, and energization of tonoplast membrane, through utilizing the energy yielded by the aforementioned hydrolysis to perfect transportation of cytoplasmic H⁺ into vacuole. Energy generated inside and outside of the tonoplast membrane through forming pH gradient is required as driving force of other secondary transporter that is present on the tonoplast membrane.

Hence, plant H⁺-PPase plays a very important role in plants, however, it is expected that mycobacterial (Streptomyces coelicolor) H³⁰ -PPase also plays very important role. However, mycobacterial H⁺-PPase is different from plant H⁺-PPase, and physiological function and biochemical functions have been unknown in almost aspects thereof.

An example of recent studies on mycobacterial H⁺-PPase is described in Hsiao Y Y, Van R C, Hung S H, Lin H H, Pan R L., “Roles of histidine residues in plant vacuolar H(+) -pyrophosphatase, ” BiochimBiophys Acta. 2004 Feb. 15; 1608 (2-3): 190-9. In this literature, importance of 6 histidine residues that are highly conserved in tonoplast membrane H⁺-PPase was analyzed. In the procedure, histidine residues in mung bean tonoplast membrane H⁺-PPase were substituted with other amino acid residue, and the resulting variants of tonoplast membrane H⁺-PPase were analyzed. Consequently, it was suggested that the aforementioned 6 histidine residues play an important role in enzyme activity and structure formation of the tonoplast membrane H⁺-PPase.

Additionally, U.S. Pat. No. 5,204,239 discloses a biosensor comprising a lipid bilayer including an ion channel as a biosensor for quantitative analysis of an analyte. This biosensor comprises bridging anchoring molecules, the biosensor comprising a container defining a chamber having at least one wall comprised of an apolar material exposed to the containment chamber; a bulk aqueous electrolyte medium contained in the chamber; a reference electrode located in an upper part of the chamber immersed in the electrolyte medium; a recording electrode located at the bottom of the chamber; a liquid crystalline membrane comprised of a lipid bilayer doped with ion channels, wherein the liquid crystalline membrane is immersed in the electrolyte medium between the reference electrode and the recording electrode; and bridging anchoring molecules attached to the recording electrode on one side and to the lipid bilayer on the other side to anchor the lipid bilayer to the recording electrode in a spaced relationship so that the lipid bilayer is in continuous contact with the bulk aqueous electrolyte medium on both the upper and lower surfaces of the lipid bilayer with the boundaries of the lipid bilayer being sealed by apolar contact with the apolar material of the at least one wall.

As described in the foregoings, some methods have been conventionally known as techniques for the measurement of PPi, however, any of the methods is disadvantageous in high cost because multiple kinds of enzymes, reagents and the like are required, and also, in complicated steps. Furthermore, all the enzymes employed are unstable to heat, therefore, they must be stored in ice ad libitum during the use.

When the enzyme used in the measurement of PPi has heat resistance as described below, disadvantages in the aforementioned conventional technique shall be greatly resolved. Specifically, even in the case of being exposed under a condition of at least at 40° C. for 30 minutes, similar activity is retained to that in the case of being stored in ice for 30 minutes. However, among the enzymes used in the measurement of PPi, such enzyme having heat resistance is not known.

Also, in the conventional method of detecting a nucleic acid base sequence or a base type, a process for detecting PPi in which PPi is converted into ATP, and thereafter a luciferase reaction is utilized, has been often used in light of the sensitivity and the like. However, in this instance, DATP generally used in the primer extension reaction can not be used because it becomes a substrate for the luciferase reaction. Therefore, it is disadvantageous in that use of a particular DATP analogue in place of DATP is required which acts as a substrate for DNA polymerase, and does not act as a substrate for the luciferase reaction.

Moreover, plant H⁺-PPase is inactivated upon contact with a Tris buffer. Thus, when the solution to be a subject of the measurement contains a Tris buffer, problem of impossible measurement of PPi using plant H⁺-PPase was also present.

### SUMMARY OF THE INVENTION

The present inventors elaborately investigated with respect to the above problems, and as a consequence, found that mycobacterial H⁺-PPase has heat resistance among H⁺-PPases, and that it is not inactivated even though it is brought into contact with a Tris buffer. Thus, the present invention was accomplished.

Accordingly, the present invention was made on the basis of the above findings, and an object of the present invention is to provide a method of measuring PPi and a method of detecting a primer extension reaction, and a device for performing the same.

Specifically, the present invention provides a method of measuring pyrophosphate which comprises:

step (a) of adding a solution comprising pyrophosphate into a first region among the first region and a second region defined by a membrane that retains mycobacterial H⁺-PPase and that is H⁺ impermeable such that the solution is brought into contact with the membrane; and

step (b) of measuring H⁺ concentration of either one of the first region or the second region following the step (a),

wherein the active site that hydrolyzes pyrophosphate of the aforementioned mycobacterial H⁺-pyrophosphatase is exposed to the first region.

The aforementioned solution may comprise a Tris buffer.

In the aforementioned step (b), the H⁺ concentration of either one of the first region or the second region may be optically measured.

In the aforementioned step (b), a pH sensitive dye or a membrane potential sensitive dye may be added to at least one of the aforementioned first region or the aforementioned second region, and the H⁺ concentration may be measured by analysis of an optical characteristic of the aforementioned pH sensitive dye or the membrane potential sensitive dye.

The aforementioned pH sensitive dye or the membrane potential sensitive dye is preferably at least one of the group consisting of pyranin, fluorescein isothiocyanate-dextran, acridine orange, quinacrine and oxonol V.

In the aforementioned step (b), the H⁺ concentration of either one of the aforementioned first region or the aforementioned second region may be electrically measured.

The aforementioned mycobacterium is preferably Streptomyces coelicolor.

Also, the present invention provides a device for measuring pyrophosphate which comprises:

a container,

a H⁺ impermeable membrane that defines the aforementioned container into an internal region and an external region;

a reference electrode provided so that it is brought into contact with a solution reserved in the aforementioned external region or internal region; and

a H⁺ sensitive electrode provided so that it is brought into contact with a solution reserved in the aforementioned internal region,

wherein the aforementioned membrane has an active site that hydrolyzes pyrophosphate of mycobacterial H⁺-pyrophosphatase such that it is exposed to the aforementioned external region.

The aforementioned solution may comprise a Tris buffer.

The aforementioned mycobacterium is preferably Streptomyces coelicolor.

Furthermore, the present invention provides a method of detecting a primer extension reaction using the method of measuring pyrophosphate described above,

said method comprising, prior to the aforementioned step (a), a step (c) of preparing a reaction solution which comprises a subject nucleic acid and a primer having a base sequence that complementarily binds to the subject nucleic acid, and produces pyrophosphate when the extension reaction of the aforementioned primer occurs,

wherein in the aforementioned step (a), the aforementioned reaction solution that will contain pyrophosphate produced upon occurrence of the extension reaction of the primer in the aforementioned step (c) is added to the aforementioned first region such that it is brought into contact with the aforementioned membrane, and the presence of a particular base sequence or base type in the aforementioned subject nucleic acid is discriminated by measuring pyrophosphate in the aforementioned reaction solution.

In the aforementioned step (b), the H⁺ concentration may be optically measured.

In the aforementioned step (b), a pH sensitive dye or a membrane potential sensitive dye may be added to at least one of the aforementioned first region or the aforementioned second region, and the H⁺ concentration may be measured by analysis of an optical characteristic of the aforementioned pH sensitive dye or the membrane potential sensitive dye.

The aforementioned pH sensitive dye or the membrane potential sensitive dye is preferably at least one of the group consisting of pyranin, fluorescein isothiocyanate-dextran, acridine orange, quinacrine and oxonol V.

In the aforementioned step (b), the H⁺ concentration of at least one of the aforementioned first region or the aforementioned second region may be electrically measured.

Also, the present invention provides a device for detecting a primer extension reaction

which comprises the device for measuring pyrophosphate described above, wherein

the aforementioned container is a reaction vessel comprising a sample inlet for injecting a sample,

a primer extension reaction tank for carrying out a treatment of a primer extension reaction,

a pyrophosphate reaction tank for allowing a reaction for the measurement of pyrophosphate, and

a flow path for connecting the aforementioned primer extension reaction tank and the pyrophosphate reaction tank,

wherein the aforementioned primer extension reaction tank reserves a reaction solution which is a solution containing a nucleic acid, and a primer having a base sequence comprising a complementary binding region that complementarily binds to the nucleic acid, and which produces pyrophosphate when an extension reaction of the aforementioned primer occurs,

the aforementioned pyrophosphate reaction tank has a detection device for detecting a signal generated within the tank, by the reference electrode and H⁺ sensitive electrode, and

wherein a solution containing pyrophosphate produced by the primer extension reaction is added such that it is brought into contact with the aforementioned first region among the first region and the second region defined by a membrane that retains mycobacterial H⁺-pyrophosphatase and that is H⁺ impermeable, and thereafter, H⁺ concentration of either one of the aforementioned first region or the aforementioned second region is measured.

It is preferred that the aforementioned device for detecting a primer extension reaction further comprises a temperature regulation means for regulating the temperature of the aforementioned primer extension reaction tank.

It is preferred that the aforementioned device for detecting a primer extension reaction further comprises an analysis means for analyzing the measurement result in the aforementioned detection device.

As is shown in FIG. 1, H⁺-PPase is intrinsically present in lipid bilayers such as tonoplast membrane and the like in nature, and has an active site, which hydrolyzes PPi, in the form of being exposed to either one side of two regions defined by this membrane. When PPi is present in the region at the side where the PPi hydrolyzing active site is exposed, H⁺-PPase has a property to hydrolyze this PPi into phosphoric acid, as well as to transport H⁺ within the region at the side where the PPi hydrolyzing active site is exposed, toward the opposite side region definedbythemembrane. Thus, the enzyme reaction of H⁺-PPase reduces H⁺ concentration in the region at the side where the PPi hydrolyzing active site of H⁺-PPase is exposed, among the two regions defined by the membrane, while H⁺ concentration in another region is increased.

According to the method of measuring PPi of the present invention, H⁺ is transported from the first region to the second region by allowing a solution containing PPi to be reserved in the aforementioned first region such that it is brought into contact with the membrane, among the first region and second region defined by a membrane that retains mycobacterial H⁺-pyrophosphatase and that is H⁺ impermeable, thereby altering the H⁺ concentration of the first solution and second solution. Accordingly, amount of PPi in the first solution can be determined by measuring the alteration in H⁺ concentration of either one of the first solution or the second solution. Therefore, the method of measuring PPi of the present invention does not necessitate multiple kinds of enzymes, reagents and the like, with simple steps to enable reduction in costs for the measurement.

Also, because the heat resistant mycobacterial H⁺-PPase has its enzyme activity even at a temperature of 50° C. or higher unlike plant H⁺-PPase, strict temperature regulation is not required contrary to conventional techniques for measuring PPi.

H⁺-PPase derived from mycobacteria or thermophile is easy to deal with because enzyme activity is retained at 60° C. or higher. Further, mycobacterial H⁺-PPase is more preferred because the present inventor established a means for mass production.

Moreover, in the device for measuring PPi of the present invention, upon injection of a sample solution into the container, when PPi is present in the sample solution, H⁺-PPase enzyme reaction occurs thereby increasing the H⁺ concentration in the internal region defined by the membrane, while reducing the H⁺ concentration in the external region. Accordingly, amount of PPi can be quantitatively determined by electrically measuring the alteration of the H⁺ concentration with the reference electrode and the H⁺ sensitive electrode.

As a method of discriminating a base type of a particular base present in a nucleic acid, for example, there exists a method of discriminating a base type of a base to which discrimination is intended, the method comprising allowing a primer extension reaction using a primer having a sequence that is completely complementary to a base sequence adjacent to 3′ of the base to which discrimination is intended, and dNTP that is complementary to an expected base type of the base to which discrimination is intended, then discrimination is executed based on the extent of progress of the primer extension reaction. Further, there also exists a method in which a so called allele specific primer is used, which has a base sequence that is complementary to the base sequence containing the base to which discrimination is intended, and which yields the difference in extent of the progress of the primer extension reaction, dependent on the base type of the base to which discrimination is intended, when the primer extension reaction is carried out concomitantly using 4 kinds of dNTPs.

All methods are common in respect of discrimination of a particular base sequence or base type based on the extent of progress of the primer extension reaction. The primer hybridizes to a nucleic acid having a complementary base sequence, and is extended by a primer extension reaction. When the primer extension reaction is caused, PPi is produced. The method of detection and the device for detection of a particular base sequence of the present invention can analyze the extent of progress of the primer extension reaction by measuring PPi produced by the primer extension reaction. Therefore, base type of a particular base can be discriminated.

Moreover, in case where discrimination of the presence/absence of a nucleic acid having a particular base sequence in a sample solution is intended, presence of the nucleic acid having a base sequence that is complementary to the primer in the solution is proven when the primer extension reaction proceeds. To the contrary, when the primer extension reaction does not proceed, absence of a nucleic acid having a base sequence that is complementary to the primer in the solution is proven.

Hence, it is also possible that the method of detection and the device for detection of a particular base sequence of the present invention discriminate the presence/absence of a nucleic acid having a particular base sequence in a sample solution, and detect a particular nucleic acid.

According to the present invention, use of the mycobacterial H⁺-PPase enables to provide a method of quantitative determination of PPi with only one kind of enzyme, and without need of strict temperature regulation, contrary to conventionally known methods of quantitative determination of PPi.

Also, according to the present invention, use of the mycobacterial H⁺-PPase enables to provide a method of detecting a primer extension reaction with fewer kinds of enzymes to be used in comparison with conventional methods of detecting a primer extension reaction, and contrary to the conventional methods of detecting a primer extension reaction, general DATP can be used without need of strict temperature regulation.

In addition, according to the present invention, use of the mycobacterial H⁺-PPase enables quantitative determination of PPi even though the solution to be a subject of measurement contains a Tris buffer.

### BRIEF DESCRIPTION OF THE DRAWINGS

FIG. 1 is a conceptual view illustrating H⁺-PPase;

FIG. 2 is an explanatory view for illustrating a principle of the method of measuring PPi according to Embodiment 1;

FIG. 3 is a view illustrating a PPi measurement kit according to Embodiment 2;

FIG. 4 is a view illustrating one example of an optical device for measuring PPi according to Embodiment 3;

FIG. 5 is a view illustrating one example of an electrical device for measuring PPi according to Embodiment 4;

FIG. 6 is a view illustrating another example of an electrical device for measuring PPi according to Embodiment 4;

FIG. 7 is a view illustrating still another example of an electrical device for measuring PPi according to Embodiment 4;

FIG. 8 is a view illustrating yet another example of an electrical device for measuring PPi according to Embodiment 4;

FIG. 9 is an explanatory view for illustrating a principle of the method of detecting a primer extension reaction according to Embodiment 5;

FIG. 10 is a view illustrating one example of a device for detecting a primer extension reaction according to Embodiment 5; FIG. 10A illustrates a type with a horizontal reaction vessel; and FIG. 10B illustrates a type with avertical reaction vessel, respectively;

FIG. 11 is an explanatory view for illustrating a method of the experiment for analyzing thermostability of mycobacterial H⁺-PPase. In this experiment, a basic buffer for measurement in the step S3 is 20 mM Bicine-NaOH, pH 8.0, 100 mM KCl, 1 mM MgCl₂, 0.15 M sucrose, 0.4 mM Na₄PPi. However, in the experiment of Example 3, a 20 mM buffer suited for each pH was used in place of 20 mM Bicine-NaOH, pH 8.0. Furthermore, as a coloring liquid in the step S5, “PhosphaC Test Wako (trade name)” manufactured by Wako Pure Chemical Industries, Ltd. was used;

FIG. 12 is a view illustrating experimental results of analysis of thermostability of mycobacterial H⁺-PPase. In this Figure, “A” is a view showing a membrane sample of Escherichia coli intrinsically including mycobacterial H⁺-PPase, and “B” is a view showing a purified mycobacterial H⁺-PPase sample;

FIG. 13 is a view illustrating a method of the experiment of inhibition of the enzyme activity of mycobacterial H⁺-PPase by Tris-based buffers. In this experiment, basic buffer for measurement in the step S12 is 0-100 mM Tris-HCl, pH 7.3, 50 mM or 0 mM KCl, 1 mM MgCl₂, 0.15 M sucrose, 0.4 mM Na₄PPi. However, in the experiment of Example 3, a 20 mM buffer suited for each pHwas used in place of 0-100 mM Tris-HCl, pH 7.3. Furthermore, as a coloring liquid in the step S14, “PhosphaC Test Wako (trade name)” manufactured by Wako Pure Chemical Industries, Ltd. was used; and

FIG. 14 is a view illustrating experimental results of inhibition of the enzyme activity of mycobacterial H⁺-PPase by Tris-based buffers. In this Figure, “A” is a view showing a membrane sample of Escherichia coli intrinsically including mycobacterial H⁺-PPase, and “B” is a view showing a membrane sample of mung bean (Vigna radiata) tonoplast.

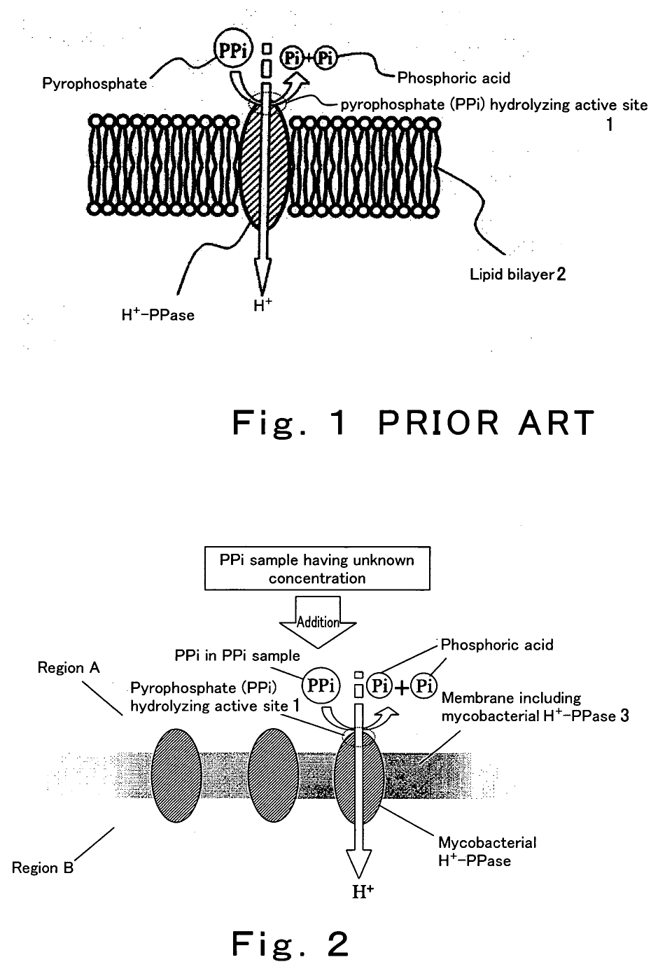

### DETAILED DESCRIPTION OF THE INVENTION

Hereinafter, preferred Embodiments of the present invention are explained with reference to accompanying drawings.

Herein, enzyme “which retains similar activity to that in the case of being stored in ice for 30 minutes, even if it is exposed under a condition of at least at 40° C. for 30 minutes” is defined as “thermotolerant” enzyme.

First, thermostability and chemical stability of mycobacterial H⁺-PPase found by the present inventors will be explained.

<Experiment for Analyzing Thermostability of Mycobacterial H⁺-PPase>

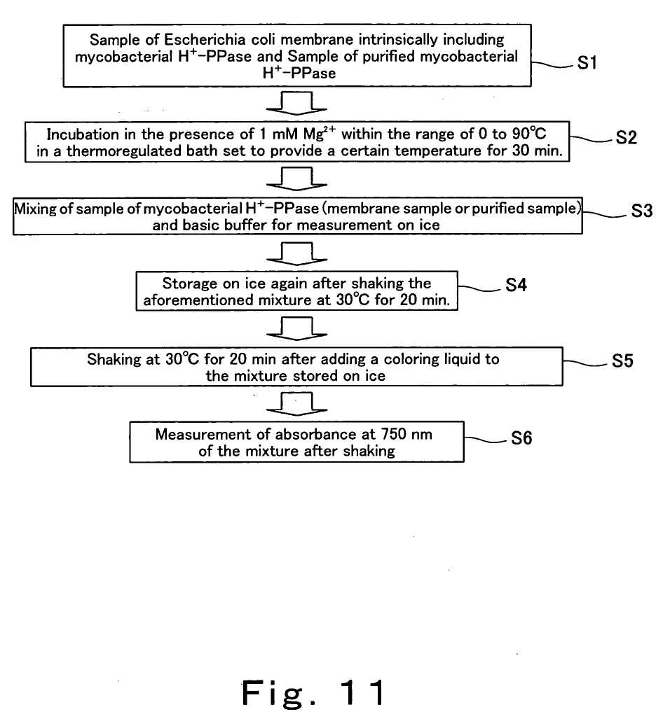

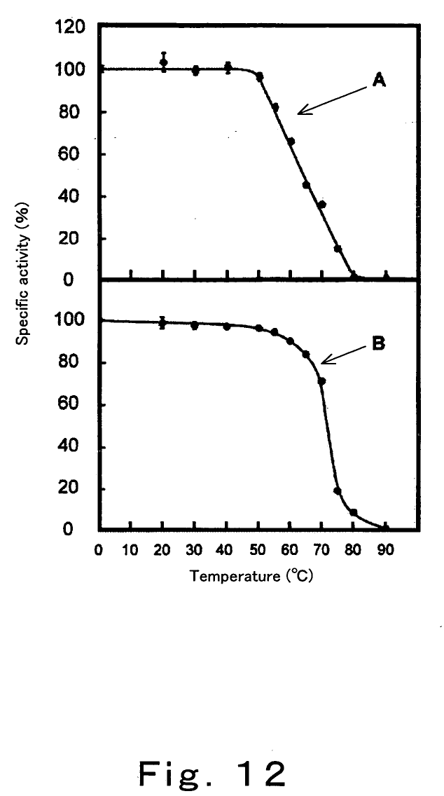

At the beginning, analysis of thermostability of mycobacterial H⁺-PPase was carried out. First, an Escherichia coli strain allowed to express mycobacterial H⁺-PPase within the membrane was produced, and membrane fraction of this Escherichia coli was prepared. Hereinafter, this membrane fraction is referred to as Escherichia coli membrane intrinsically including mycobacterial H⁺-PPase. Then, this Escherichia coli membrane intrinsically including mycobacterial H⁺-PPase was solubilized with CHAPS, and purified by a sucrose density gradient centrifugation method to prepare purified mycobacterial H⁺-PPase. These Escherichia coli membrane intrinsically including mycobacterial H⁺-PPase sample and purified mycobacterial H⁺-PPase sample were subjected to the experiment in accordance with the method illustrated in FIG. 11. The results are shown in FIG. 12.

In FIG. 12, specific activity based on the enzyme activity yielded following incubation at 0° C., which is assumed to be 100%, is presented in the ordinate, while the incubation temperature is presented in the abscissa. From the curves A and B shown in FIG. 12, it was revealed that both samples exhibited potent thermostability, i.e., 100% activity was retained following incubation up to 50° C., and greater than 60% activity was also retained following incubation up to 60° C.

<Experiment of Inhibition of Enzyme Activity of Mycobacterial H⁺-PPase by Tris-Based Buffer>

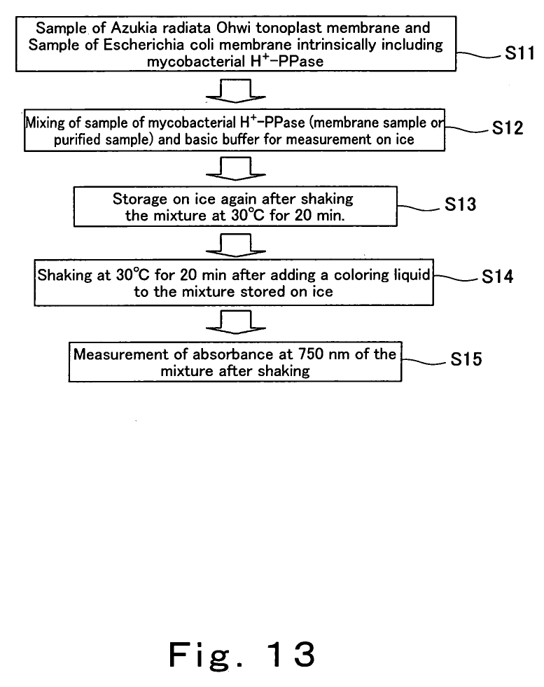

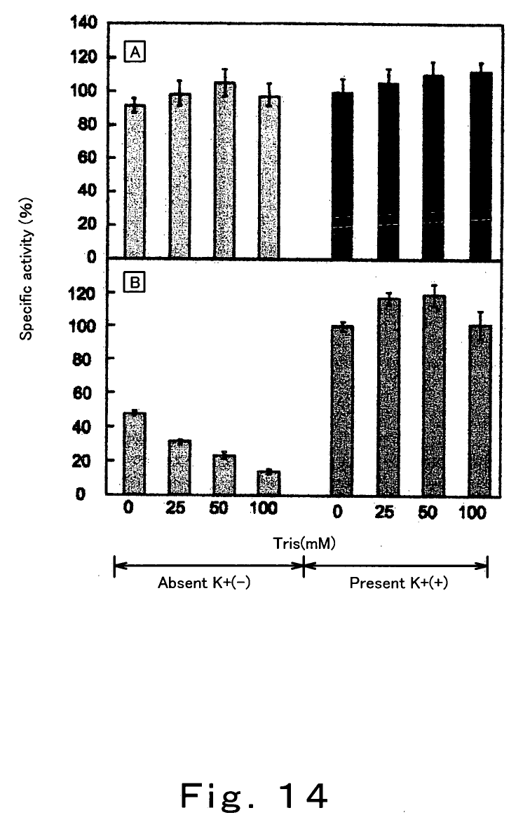

Next, as an analysis in connection with chemical stability of mycobacterial H⁺-PPase, influences on conventional H⁺-PPase and mycobacterial H⁺-PPase by a Tris-based buffer were compared. More specifically, according to the method illustrated in FIG. 13, influences by 0-100 mM Tris-HCl (pH7.3) on hydrolyzing activity of mung bean (Vigna radiata) H⁺-PPase and mycobacterial H⁺-PPase in the presence of 50 mM K⁺(K⁺(+)) and in the absence thereof (K⁺ (−)) were compared. The results are shown in FIG. 14.

In FIG. 14, specific activities for both of the mung bean H⁺-PPase and mycobacterial H⁺-PPase, based on the activity yielded in the presence of 50 mM K⁺ and in the absence of Tris, which is assumed to be 100%, are presented in the ordinate. From this Figure, it was elucidated that although the activity of mung bean H⁺-PPase is significantly inhibited by Tris-HCl particularly in the absence of K⁺, mycobacterial H⁺-PPase was not inhibited by Tris-HCl at all irrespective of the presence and absence of K⁺.

As in the foregoings, it is understood from the above experiments that mycobacterial H⁺-PPase has heat resistance, and is not inactivated even though it is brought into contact with a Tris buffer.

Hereinafter, Embodiments of the present invention are sequentially explained with reference to accompanying drawings. The present invention is not anyhow limited thereto.

(Embodiment 1)

Embodiment 1 demonstrates a method of quantitative determination of PPi using mycobacterial H⁺-PPase. Explanation will be made below with reference to FIG. 2.

First, a state involving a membrane intrinsically including mycobacterial H⁺-PPase, and two kinds of regions (region A (first region) and region B (second region) in FIG. 2) defined by the membrane is created. The membrane for use in this state may be one that retains the enzyme activity of mycobacterial H⁺-PPase without significant suppression, and is almost H⁺ impermeable. For example, the membrane may be a natural or artificial lipid bilayer, or any one else. Also, the shape may be either in the shape of so called endoplasmic reticulum or in the plane shape, as long as it defines the two regions as described above.

Orientation of the mycobacterial H⁺-PPase that is intrinsically included in this membrane is preferably uniform in light of sensitivity of the PPi measurement, however, those with different orientation may be present admixed. Further, the two regions described above may be previously filled with a type of a solution such as a buffer, or may be in a wet state to the extent that the aforementioned membrane structure and the activity of mycobacterial H⁺-PPase are not completely lost.

Next, a PPi sample having an unknown concentration is added to one side of the aforementioned two regions (region A side in FIG. 2). Upon this operation, to the aforementioned region on one side must be exposed entire or a part of the mycobacterial H⁺-PPase. This operation allows PPi in the PPi sample to be hydrolyzed, thereby causing transport of H⁺ from the aforementioned region on one side toward the region on another side. Because this H⁺ transport is carried out depending on the PPi concentration in the aforementioned PPi sample, analysis of the same enables measurement of the PPi concentration in the aforementioned PPi sample.

Examples of method of analyzing H⁺ transport include optical methods and electrical methods. When the optical method is used, for example, pH in either one of the above two regions after the H⁺ transport may be examined with pH test paper, or a substance having an optical characteristic that can be changed depending on alteration of the H⁺ concentration may be added to either one of the above two regions.

Specific examples of the substance having an optical characteristic that can be changed depending on alteration of the H⁺ concentration include pH sensitive dyes or membrane potential sensitive dyes. Among these, pyranin, fluorescein isothiocyanate-dextran, acridine orange, quinacrine or oxonol V is preferred in terms of easy handling and the like.

Furthermore, examples of the electrical method include metal electrode methods (hydrogen electrode method, quinhydrone electrode method, antimony electrode method and the like), a glass electrode method, an ISFET electrode method, a patch clamp method, a LAPS method, lipid-soluble selective electrode methods in which an lipid-soluble ion (specifically, tetraphenyl phosphonium, triphenylmethyl phosphonium, ClO⁴⁻, tetraphenyl boron or the like) and a lipid-soluble ion selective electrode are used in combination, and the like. However, the method of analyzing H⁺ transport is not limited to these methods, but any method which can convert H⁺ transport into an optical or electrical signal, and detect the signal is permitted.

(Embodiment 2)

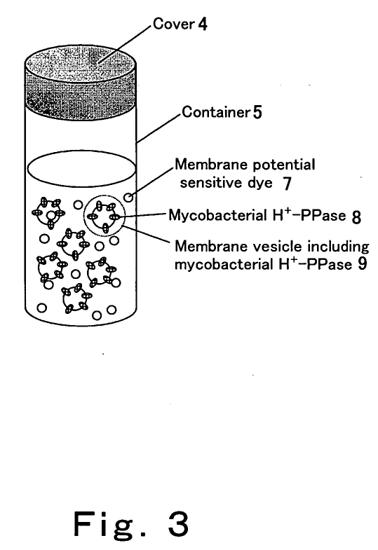

Embodiment 2 demonstrates a kit for use in the method of measuring PPi (PPi measurement kit). Explanation will be made below with reference to FIG. 3.

In FIG. 3, a state of a solution containing a PPi measurement kit of this Embodiment is stores in a container is illustrated. The PPi measurement kit of this Embodiment is constituted from at least a membrane vesicle 9 intrinsically including mycobacterial H⁺-PPase and a pH sensitive dye 6 or membrane potential sensitive dye 7. Therefore, the user mixes a PPi sample having an unknown concentration with the PPi measurement kit of this Example, thereby capable of measuring the PPi concentration in the unknown sample by detecting and analyzing the optical signal of the pH sensitive dye 6 or membrane potential sensitive dye 7 after mixing.

In this procedure, the membrane vesicle 9 including the mycobacterial H⁺-PPase shown in FIG. 3 may be, for example, either a natural or artificial lipid bilayer, or may be any one else as long as it retains the enzyme activity of mycobacterial H⁺-PPase 8 without significant suppression, and is almost H⁺ impermeable.

Also, entire or a part of the mycobacterial H⁺-PPase 8 must be in the state of being exposed to outside of the membrane vesicle 9.

Type of the pH sensitive dye 6 is not limited as long as its optical characteristic is changed depending on alteration of the H⁺ concentration of the solution inside or outside of the membrane vesicle 9, however, it is preferably pyranin, fluorescein isothiocyanate-dextran, acridine orange or quinacrine in terms of easy handling and the like.

Type of the membrane potential sensitive dye 7 is not limited as long as its optical characteristic is changed depending on the membrane potential of the membrane vesicle 9, however, it is preferably oxonol V in terms of easy handling and the like.

The membrane vesicle 9, and the pH sensitive dye 6 or the membrane potential sensitive dye 7 may be provided to the user in a dissolved state in a solvent such as a buffer as shown in FIG. 3, or alternatively, may be dissolved in a solvent such as a desired buffer by the user immediately before use. They may be provided to the user such that it can be present in membrane vesicle 9 in a state to allow the membrane vesicle to be properly formed upon quantitative determination of PPi, and that the activity of the mycobacterial H⁺-PPase is retained.

Further, the membrane vesicle 9 and the pH sensitive dye 6 or the membrane potential sensitive dye 7 may be provided to the user in a state stored within a sealed container after previously mixing as shown in FIG. 3, or alternatively, they may be provided to the user in a state stored separately in different sealed containers, and mixed before use by the user.

(Embodiment 3)

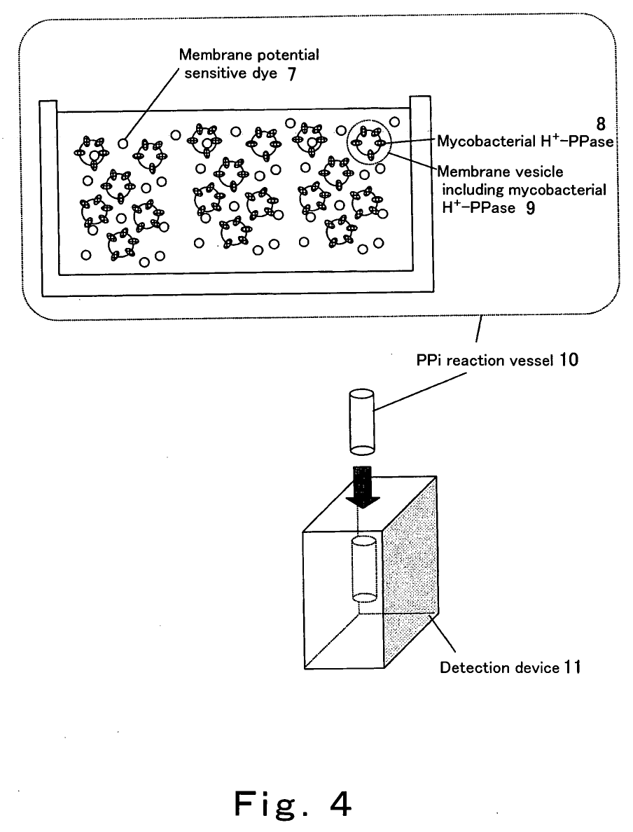

Embodiment 3 demonstrates one example of the optical device for measuring PPi in which mycobacterial H⁺-PPase is used. Explanation will be made below with reference to FIG. 4.

The device for measuring PPi of this Embodiment is an optical device for measuring PPi in which mycobacterial H⁺-PPase is used, and has a PPi reaction vessel 10 for carrying out a reaction for measuring PPi in a PPi sample having an unknown concentration, and a detection device 11 for detecting an optical signal in this PPi reaction vessel.

More specifically, the PPi reaction vessel 11 includes a mixture of at least an endoplasmic reticulum-like membrane intrinsically including mycobacterial H⁺-PPase (membrane vesicle 9 including mycobacterial H⁺-PPase), and a pH sensitive dye 6 or a membrane potential sensitive dye 7. Entire or a part of the mycobacterial H⁺-PPase must be in the state of being exposed to outside of the membrane vesicle. Further, the detection device 11 is constructed such that the PPi reaction vessel is detachable, and detection of the optical signal of the pH sensitive dye 6 or the membrane potential sensitive dye 7 can be perfected in the attached state.

When the user adds a PPi sample having an unknown concentration into the PPi reaction vessel 10, PPi in the PPi sample is hydrolyzed by the mycobacterial H⁺-PPase 8, and with this event, H⁺ transport is carried out from outside toward inside of the membrane vesicle 9. As a result, the pH sensitive dye 6 or the membrane potential sensitive dye 7 exhibits an optical signal depending on the H⁺ transport, and accordingly, analysis of the signal with a detection device 11 enables measurement of the PPi concentration in the PPi sample.

In addition, the membrane vesicle 9 may be, for example, either a natural or artificial lipid bilayer, or may be any one else as long as it retains the enzyme activity of mycobacterial H⁺-PPase 8 without significant suppression, and is almost H⁺ impermeable.

Mixture of the membrane vesicle 9 and the pH sensitive dye 6 or the membrane potential sensitive dye 7 may be in a solution state obtained by dissolving in any solvent such as a buffer, or may be in a wet state to the extent that activities of the membrane vesicle 9 and the mycobacterial H⁺-PPase 8 are not completely lost.

Type of the pH sensitive dye 6 is not limited as long as its optical characteristic is changed depending on alteration of the H⁺ concentration of the solution inside or outside of the membrane vesicle 9, however, it is preferably pyranin, fluorescein isothiocyanate-dextran, acridine orange or quinacrine in terms of easy handling and the like.

Type of the membrane potential sensitive dye 7 is not limited as long as its optical characteristic is changed depending on the membrane potential of the membrane vesicle 9, however, it is preferably oxonol V in terms of easy handling and the like.

The PPi reaction vessel 10 is preferably sealed with a cover or the like. More specifically, it is preferred that the user opens the cover before use, and a PPi sample is added into the PPi reaction vessel.

(Embodiment 4)

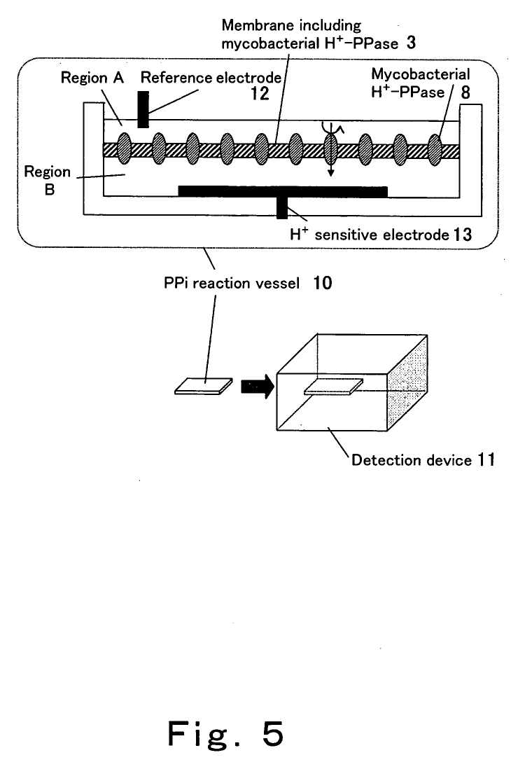

Embodiment 4 demonstrates one example of the electrical device for measuring PPi in which mycobacterial H⁺-PPase is used. Explanation will be made below with reference to FIGS. 5 to 8.

The device for measuring PPi of this Embodiment has, as shown in FIG. 5, a PPi reaction vessel 10 for carrying out the reaction for measuring PPi concentration in a PPi sample having an unknown concentration, and a detection device 11 for detecting an electrical signal in this PPi reaction vessel. The device will be explained below in detail.

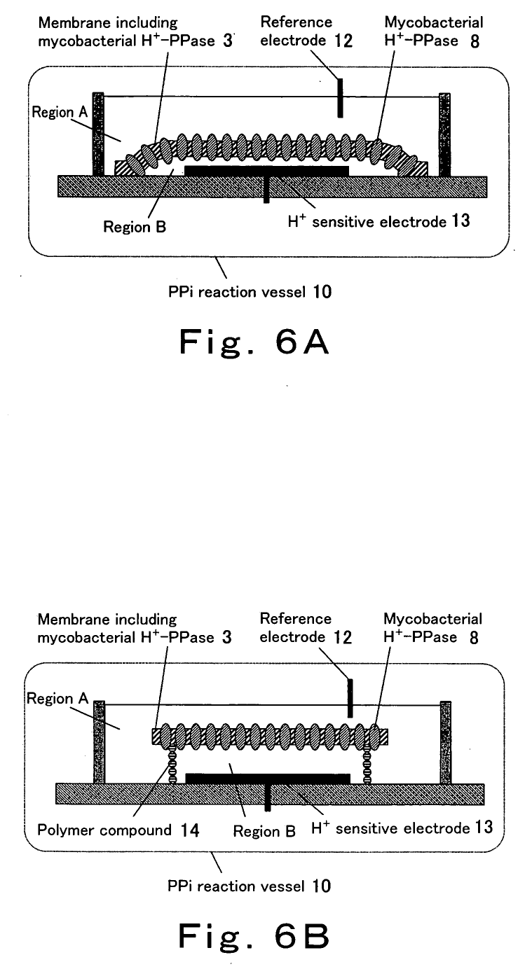

First, in FIG. 5 and FIGS. 6A and 6B, a membrane 3 including mycobacterial H⁺-PPase is set in the PPi reaction vessel 10, thereby constructing two regions A and B. The membrane 13 herein may be fixed on the side face of the PPi reaction vessel 10 as shown in FIG. 5. Alternatively, it may be directly fixed on the bottom face of the PPi reaction vessel 10 as shown in FIG. 6A, or may be fixed on the bottom face of the PPi reaction vessel 10 via a polymer compound 14 such as a linear carbon compound or the like as shown in FIG. 6B.

Entire or a part of the mycobacterial H⁺-PPase 8 must be in the state of being exposed to the region A side. Moreover, in every case shown in FIG. 5 or FIGS. 6A and 6B, a H⁺ sensitive electrode 13 is disposed on the bottom of the PPi reaction vessel 10 such that it is brought into contact with the region B, while a reference electrode 12 corresponding to this H⁺ sensitive electrode 13 is disposed on the region A side. In the construction, electric potential difference between these electrodes can be analyzed with the detection device. Although the reference electrode 12 is disposed on the region A side in FIG. 5 and FIGS. 6A and 6B, it may be disposed on the region B side so that it is not in contact with the H⁺ sensitive electrode 13.

In such a PPi reaction vessel 10, when a PPi sample having an unknown concentration is added to the region A side, the PPi hydrolyzing site, which is exposed to the region A side, of the mycobacterial H⁺-PPase 8 hydrolyzes PPi in this sample, and with this event, H⁺ transport is carried out from the region A side toward the B side. Then, alteration in the H⁺ concentration on the region B side can be determined by analyzing the change in electric potential of the H⁺ sensitive electrode 13, and the H⁺ concentration in the region B side following addition of the PPi sample is dependent on the PPi concentration in this PPi sample. Therefore, measurement of the PPi concentration in a PPi sample is enabled by analyzing the electric potential of the H⁺ sensitive electrode 13 following addition of the PPi sample with the detection device.

Upon the measurement, any state of the regions A and B may be acceptable as long as they are filled with a solution such as a buffer or the like. The regions A and B may be previously filled with a solution to provide to the user, or the user may fill in the regions A and B with the solution before use.

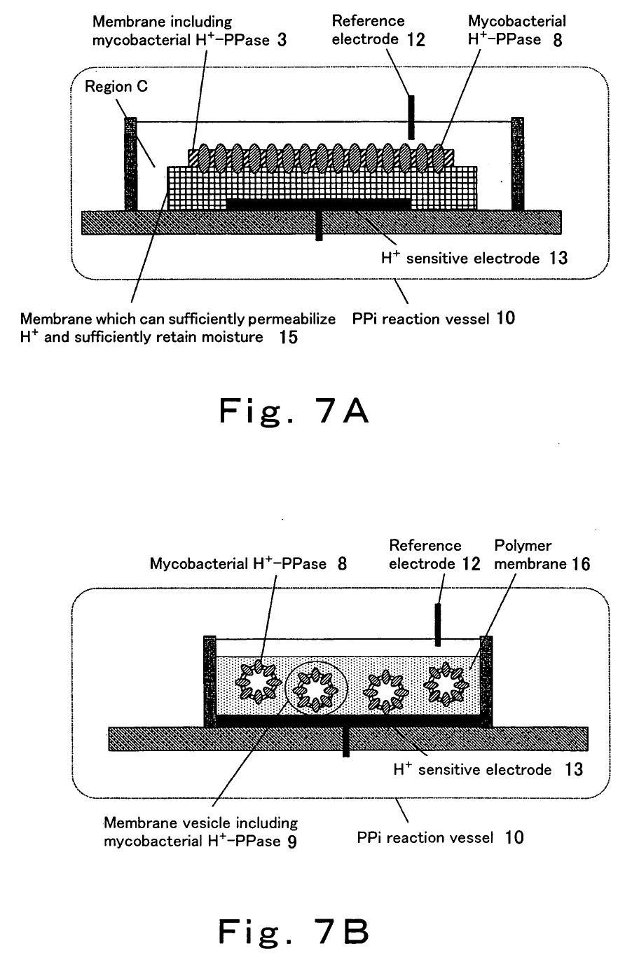

Also, another example of the embodiment of the PPi reaction vessel 10 is illustrated in FIG. 7A. More specifically, a membrane 15 which sufficiently permeabilizes H⁺ and which can sufficiently retain moisture may be formed on a H⁺ sensitive electrode 13 disposed on the bottom face of the PPi reaction vessel 10, and further, the membrane 3 including mycobacterial H⁺-PPase may be fixed on the surface thereof.

As the membrane 15 which sufficiently permeabilizes H⁺ and which can sufficiently retain moisture, a membrane comprising polymer gel such as agarose gel, or a fullerene-like compound, or the like may be used. Entire or a part the mycobacterial H⁺-PPase 8 must be in the state of being exposed to the region C side that is not in contact with the membrane 15 which sufficiently permeabilizes H⁺ and which can sufficiently retain moisture. The PPi sample is also added to this region C. Consequently, the mycobacterial H⁺-PPase 8 whose PPi hydrolyzing active site is exposed to the region C hydrolyzes PPi in the PPi sample, and with this event, H⁺ is transported from the region C toward the membrane 15 which sufficiently permeabilizes H⁺ and which can sufficiently retain moisture. Because amount of the transported H⁺ is dependent on the PPi concentration in the PPi sample, and thus transported H⁺ can reach onto the H⁺ sensitive electrode 13, the PPi concentration in the PPi sample can be measured with the H⁺ sensitive electrode 13.

Also, another example of the embodiment of the PPi reaction vessel 10 is illustrated in FIG. 7B. More specifically, a membrane vesicle 9 is used as a membrane including mycobacterial H⁺-PPase in FIG. 7B. The membrane vesicle 9 herein can be, for example, fixed on the surface of the H⁺ sensitive electrode 13 by means of a polymer membrane 16. In this case, the membrane for use in fixing of the mycobacterial H⁺-PPase 8 is preferably a membrane which rapidly permeabilizes H⁺ .

When PPi is present in the PPi sample solution in the reaction vessel 10 (sensor) produced accordingly, PPi is hydrolyzed into phosphoric acid by the H⁺-PPase activity, accompanied by increase in the H⁺ concentration of the liquid internally included in the membrane vesicle 9, while the H⁺ concentration around the membrane vesicle 9 is decreased. Because the extent of this decrease in H⁺ concentration is dependent on the PPi concentration in the PPi sample, the PPi concentration in the sample solution can be measured by determination of the decrease in the H⁺ concentration with the H⁺ sensitive electrode 13 when the membrane vesicle 9 is present in close vicinity of the H⁺ sensitive electrode 13.

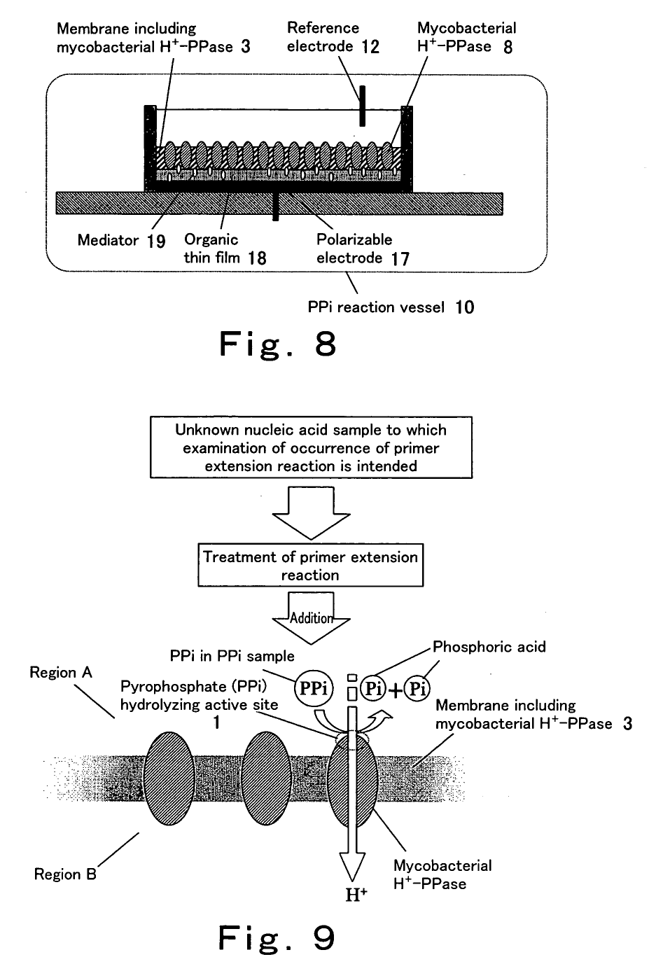

Furthermore, another example of the embodiment of the PPi reaction vessel 10 is illustrated in FIG. 8. In FIG. 8, a polarizable electrode 17 is formed on an insulating substrate, thereby also enabling amperometric measurement. Examples of the polarizable electrode 17 which may be used include those which can be used in general electrochemical measurement such as gold, platinum, carbon and the like. On the surface of this polarizable electrode 17 is formed an organic thin film 18 including a mediator 19. As the organic thin film 18, for example, SAM (self-assembled monolayer) membrane in which linear carbon having a thiol group at one end is utilized, or the like may be used. As the mediator 19, an oxidized form of a H⁺ sensitive substance may be used. The membrane 3 including H⁺-PPase is fixed on thus formed organic thin film 18.

When the membrane 3 including H⁺-PPase is a lipid membrane, hydrophobic moieties of the organic thin film and the lipid membrane face one another, and the hydrophobic moieties of the lipid membrane form the membrane surface. The H⁺-PPase 8 is fixed inside of the membrane formed by the hydrophobic moieties of the organic thin film and the lipid membrane, however, the active site that hydrolyzes PPi of the H⁺-PPase 8 is exposed outside of the membrane 13. When PPi is present in the sample solution in the reaction vessel 10 (sensor) produced accordingly, PPi is hydrolyzed into phosphoric acid by the H⁺-PPase activity, accompanied by increase in the H⁺ concentration within the organic thin film.

When the oxidized form of the H⁺ sensitive mediator 19 is present, a reduced form of the mediator 19 is produced by an oxidation-reduction reaction. Application of electric potential which is sufficiently higher than the oxidation reduction potential of the mediator 19 to the polarizable electrode 17 permits measurement of current depending on the concentration of the reduced substance of the mediator 19. Therefore, it is possible to measure the PPi concentration in the sample solution.

In stead of the organic thin film 18 including the mediator 19, an electrolytically polymerized membrane that is electrochemically active such as poly(aniline), poly(o-phenylenediamine), poly(N-methylaniline), poly(pyrrole), poly(N-methylpyrrole), poly(thiophene) or the like can be also used. Further, it is also possible to fix a membrane vesicle including mycobacterial H⁺-PPase in an electrolytically polymerized membrane or an organic thin film 18 including a mediator 19 on the polarizable electrode 17, and to measure the oxidation-reduction current of the electrolytically polymerized membrane or the mediator accompanied by decrease in H⁺ outside of the membrane vesicle, with a polarizable electrode.

Herein, the membrane 3 including mycobacterial H⁺-PPase shown in FIGS. 5 to 8 may be, for example, either a natural or artificial lipid bilayer, or may be any one else as long as it retains the enzyme activity of mycobacterial H⁺-PPase 8 without significant suppression, and is almost H⁺ impermeable. The same is applied to the membrane vesicle 9.

The membrane 3 including mycobacterial H⁺-PPase shown in FIGS. 5 to 8 may contain a protein other than mycobacterial H⁺-PPase, however, the protein is preferably a protein which does not react with PPi, or has low reactivity therewith. When PPi in the PPi sample reacts with a protein in the membrane other than H⁺-PPase, amount of PPi that reacts with H⁺-PPase is reduced, accompanied by reduction of the amount of H⁺ transport.

Also, when a protein which does not react with PPi, and transports H⁺ via a reaction with a substance other than PPi is included in the membrane, it is preferred that the substance that reacts with the protein is scarcely included in the sample solution. For example, when ATPase is included in the membrane, it is preferred that ATP is rendered to be scarcely included in the sample solution.

The H⁺ sensitive electrode 13 shown in FIGS. 5 to 7 may be any one as long as it can function as a general pH sensor, and a glass electrode, an ISFET electrode, LAPS (Light-AddressAble Potentiometric Sensor) or the like may be used. On the other hand, a hydrogen electrode, a saturated calomel electrode, a mercury-silver oxide electrode or the like may be used as the reference electrode 12, however, in light of the easy handling and the like, a silver-silver chloride electrode is preferably used.

In the Embodiments 3 and 4, the device for measuring PPi of the present invention was explained. However, merely illustrative examples were shown in these Embodiments. Accordingly, characteristic features of the device for measuring PPi of the present invention involve hydrolysis of PPi with mycobacterial H⁺-PPase, and optical or electrical detection of H⁺ transport accompanied by this event. Thus, any construction is permitted that enables measurement of the PPi concentration.

(Embodiment 5)

Embodiment 5 demonstrates methods of detecting a primer extension reaction in which mycobacterial H⁺-PPase is used (method of detecting a base sequence of a nucleic acid and method of discriminating a base type), and a kit and a device for performing these methods. As described above, the detection of a base sequence of a nucleic acid, and the discrimination of a base type are common in respect of necessary determination of occurrence of a primer extension reaction in the end, therefore, explanation will be made below with collective reference to “method of detecting a primer extension reaction, kit and device”.

[Method of Detecting a Primer Extension Reaction in which Mycobacterial H⁺-PPase is used]

In connection with the method of detecting a primer extension reaction in which mycobacterial H⁺-PPase is used according to this Embodiment, explanation will be made with reference to FIG. 9. In this method of the detection, the method of quantitative determination of PPi in which mycobacterial H⁺-PPase is used as described in Embodiment 1 is utilized.

First, a treatment of a primer extension reaction for detecting a nucleic acid sequence or discriminating a base type is carried out. Then, the procedure of Embodiment 1 may be carried out using a sample solution after subjecting to the treatment to initiate this primer extension reaction (i.e., a sample solution after completing the primer extension reaction, or a sample solution in progress of the primer extension reaction) in place of the PPi sample having an unknown concentration, and thus resulting optical or electrical signal may be analyzed. If the primer extension reaction was executed in the aforementioned treatment of the primer extension reaction, PPi shall be included in the sample solution flowing the treatment of the primer extension reaction, and to the contrary, if the primer extension reaction was not or scarcely executed in the aforementioned treatment of the primer extension reaction, PPi shall not or scarcely be included in the sample solution following the primer extension reaction. Possible quantitative determination of PPi concentration by the method of measurement according to Embodiment 1 is as described above. Therefore, by analyzing the optical or electrical signal, analysis on whether the aforementioned primer extension reaction was executed is enabled. [Kit for Detecting Primer Extension Reaction in which Mycobacterial H⁺-PPase is Used]

Next, a kit for detecting a primer extension reaction in which mycobacterial H⁺-PPase is used according to this Embodiment will be explained. Constitution of this kit for detecting a primer extension reaction is similar to that of Embodiment 2. The treatment of the primer extension reaction for detecting a nucleic acid sequence or discriminating a base type is first carried out by the user, similarly to the method of detecting a primer extension reaction as described above. Next, a sample solution after subjecting to the treatment of this primer extension reaction may be mixed with the kit of this Embodiment, and the optical signal of the pH sensitive dye 6 or the membrane potential sensitive dye 7 included in this kit may be analyzed. Accordingly, analysis on whether the aforementioned primer extension reaction was executed is enabled.

[Device for Detecting Primer Extension Reaction in which Mycobacterial H⁺-PPase is Used]

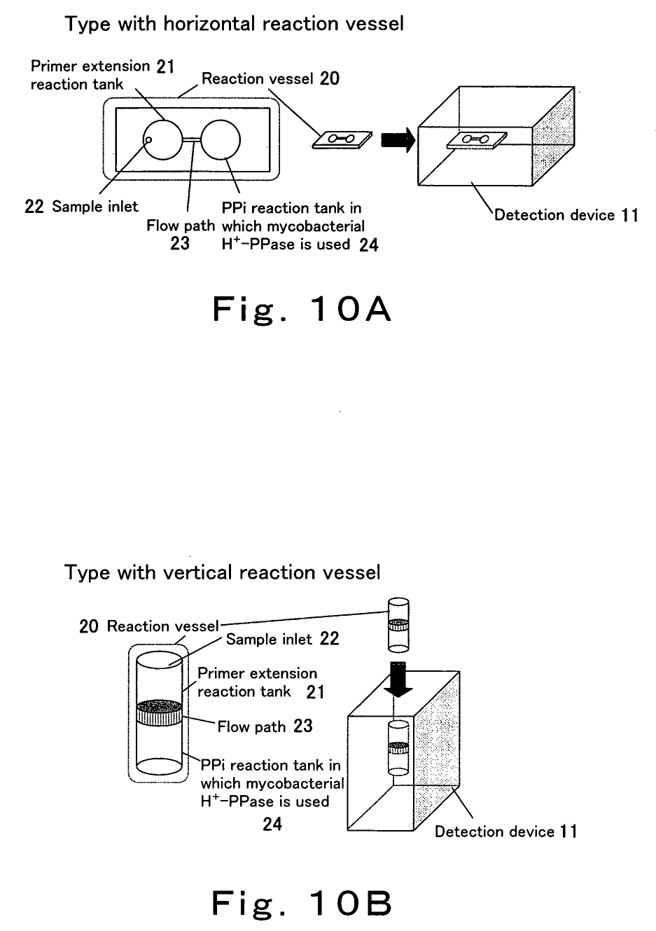

Next, explanation of the device for detecting a primer extension reaction in which mycobacterial H⁺-PPase is used according to this Embodiment will be made with reference to FIG. 10. This device for detecting a primer extension reaction comprises, as shown in FIG. 10, a reaction vassel 20 and a detection device 11. The reaction vassel 20 has a sample inlet 22 for injecting an unknown nucleic acid sample to which examination of occurrence of the primer extention reaction is intended, a primer extension reaction tank 21 for carrying out the primer extention reaction and a PPi reaction tank 24 in which the reaction for the measurement od PPi is carried out.

First, the device for detecting a primer extension reaction in which the detection device 11 is an optical detection device will be explained. The primer extension reaction tank 21 is a reaction tank for carrying out the treatment of the primer extension reaction. The PPi reaction tank 24 is a reaction tank having essentially the same function as that of the PPi reaction vessel explained in Embodiment 3. Also, the detection device 11 has a function which is similar to that of the detection device in Embodiment 3. Accordingly, it is constructed such that the optical signal in the PPi reaction tank can be detected.

More specifically, a mixture of at least an endoplasmic reticulum-like membrane (membrane vesicle) intrinsically including mycobacterial H⁺-PPase, and a pH sensitive dye 6 or membrane potential sensitive dye 7 is included in the PPi reaction tank 24, wherein entire or a part of the mycobacterial H⁺-PPase must be in the state of being exposed to outside of the membrane vesicle.

Further, the detection device 11 is constructed such that the reaction vessel 20 is detachable, and detection of the optical signal of the pH sensitive dye 6 or the membrane potential sensitive dye 7 can be perfected in the attached state.

In addition, with respect to the construction of the sample inlet 22, the primer extension reaction tank 21 and the PPi reaction tank 24, they are constructed so that after an unknown nucleic acid sample is injected from the sample inlet 22, for example, it passes a flow path 23 and the like to be first delivered to the primer extension reaction tank 21, and finally, delivered to the PPi reaction tank 24.

In the PPi reaction tank 24, PPi in the sample after subjecting to the treatment of the primer extension reaction is hydrolyzed by the mycobacterial H⁺-PPase, and with this event, H⁺ transport is carried out from outside to inside of the membrane vesicle. As a consequence, the pH sensitive dye 6 or the membrane potential sensitive dye 7 exhibits an optical signal depending on the H⁺ transport. By analyzing this signal with the detection device 11, actual execution of the primer extension reaction can be determined for the unknown nucleic acid sample.

In the primer extension reaction tank 21, all or a part of the materials which have been proven to be required in the treatment of the primer extension reaction such as polymerase, dNTP, primer and the like may be provided to the user in the state previously involved, or they may be injected from the sample inlet 22 by the user.

Moreover, the membrane vesicle may be, for example, either a natural or artificial lipid bilayer, or may be any one else as long as it retains the enzyme activity of mycobacterial H⁺-PPase without significant suppression, and is almost H⁺ impermeable.

The mixture of the membrane vesicle and the pH sensitive dye 6 or the membrane potential sensitive dye 7 may be in a solution state obtained by dissolving in any solvent such as a buffer, or may be in a wet state to the extent that the aforementioned membrane structure and activity of the mycobacterial H⁺-PPase is not completely lost.

Type of the pH sensitive dye 6 is not limited as long as its optical characteristic is changed depending on alteration of the H⁺ concentration of the solution inside or outside of the membrane vesicle, however, it is preferably pyranin, fluorescein isothiocyanate-dextran, acridine orange or quinacrine in terms of easy handling and the like.

Type of the membrane potential sensitive dye 7 is not limited as long as its optical characteristic is changed depending on the membrane potential of the membrane vesicle, however, it is preferably oxonol V in terms of easy handling and the like.

The sample inlet 22 is preferably sealed with a cover or the like. Accordingly, it is preferred that the user opens the cover before use, and an unknown nucleic acid sample is injected.

When regulation of the temperature of the primer extension reaction tank 21 is necessary for the treatment of the primer extension reaction, for example, construction with a temperature regulation function imparted to the reaction vessel 20 itself may be accepted, or construction capable of regulating the temperature in the primer extension reaction tank 21 through imparting a temperature regulation function to the detection device 11 or the like may be also accepted.

Next, explanation will be made in connection with a device for detecting a primer extension reaction in which the detection device 11 is an electrical detection device. Fundamental construction and method of use of the reaction vessel 20 in this electrical device for detecting a primer extension reaction are essentially similar to those in the optical device for detecting a primer reaction as described above.

The PPi reaction tank 24 is a reaction tank having essentially the same function as that of the PPi reaction vessel explained in Embodiment 4, and for example, it may have a structure shown in FIGS. 5 to 8. The detection device 11 has a function capable of executing a primer extension reaction in addition to the function similar to that of the detection device in Embodiment 4. More specifically, it is constructed to permit the temperature regulation, with a construction to enable detection of an electrical signal in the PPi reaction tank. Furthermore, it is constructed such that delivery of a sample after completing the primer extension reaction can be also conducted from the primer extension reaction tank to the PPi reaction tank.

First, an unknown nucleic acid sample to which examination of occurrence of a primer extension reaction is intended is injected from the sample inlet by the user, and the treatment of the primer extension reaction is carried out in the primer extension reaction tank 21. Next, this sample after subjecting to the treatment of the primer extension reaction is delivered to a region A side of the PPi reaction tank 24 (corresponding to region A in FIGS. 5 and 6). As a result, PPi concentration in the sample after subjecting to the treatment of the primer extension reaction reflects the H⁺ concentration at a region B side of the PPi reaction tank 24 (corresponding to region B in FIGS. 5 and 6), therefore, actual execution of the primer extension reaction can be determined for the unknown nucleic acid sample by analyzing with the electrical detection device 11.

In the primer extension reaction tank 21, all or a part of the materials which are required in the treatment of the primer extension reaction such as polymerase, dNTP, primer and the like may be in the state previously retained, or they may be injected from the sample inlet 22 by the user.

Furthermore, the region A and the region B of the PPi reaction tank 24 may or may not be previously filled with any solution such as a buffer or the like. In either case, the PPi concentration in an unknown sample can be electrically measured when correlation between the PPi concentration in the PPi sample to be added and the electrical signal yielded accordingly is previously comprehended.

Sealing of the sample inlet 22 with a cover or the like, and preferred construction of the temperature regulation are similar to those in the optical device for detecting a primer reaction described above.

The method of quantitative determination of PPi and the method of detecting a primer extension reaction, and the kit and the device using these methods demonstrated in Embodiments 1to 5 are characterized in that mycobacterial H⁺-PPase is used. By using the mycobacterial H⁺-PPase, multiple kinds of enzymes are not required contrary to cases of conventional quantitative determination of PPi. Also, as shown in FIG. 12, the mycobacterial H⁺-PPase has potent thermotolerance, therefore, strict temperature regulation such as placing the H⁺-PPase on ice or under a condition of 4° C. ad libitum is not required in the method of quantitative determination of PPi and the method of detecting a primer extension reaction according to Embodiments 1 and 5.

Also, among currently known H⁺-PPases, some of the enzyme activities are inhibited by a Tris-based buffer, however, such inhibition of the enzyme activity is scarcely found in mycobacterial H⁺-PPase as shown in FIG. 14. Therefore, sample preparation can be performed in a Tris-based buffer also in the method of quantitative determination of PPi and the method of detecting a primer extension reaction according to Embodiments 1 and 5. Such advantages are particularly important in respect of Embodiment 5 because a Tris-based buffer is used in many cases of the primer extension reaction as represented by PCR methods.

In addition, similar advantages to those described above are also involved in the PPi measurement kit and the kit for detecting a primer extension reaction demonstrated in Embodiments 2 and 5. More specifically, by using the mycobacterial H⁺-PPase, fewer kinds of enzymes are required than in conventional techniques, and strict temperature regulation of the kit is not necessary during use or storage because the mycobacterial H⁺-PPase is thermostable. Further, because the mycobacterial H⁺-PPase is scarcely inhibited by a Tris-based buffer for the enzyme activity, any sample prepared with a Tris-based buffer may be handled. In this respect, it is particularly important in detecting a primer extension reaction as described above.

Moreover, also in the optical and electrical devices for measuring PPi, and the optical and electrical devices for detecting a primer extension reaction demonstrated in Embodiments 3, 4 and 5, similar advantages to those described above are involved. More specifically, either one of the device for measuring PPi or the device for detecting a primer extension reaction has a reaction vessel including mycobacterial H⁺-PPase and a detection device for detecting an optical or electrical signal in this reaction vessel, however, strict temperature regulation of the reaction vessel is not necessary during use or storage because the mycobacterial H⁺-PPase is thermostable, thereby leading to easy handling.

In particular, in case of the device for detecting a primer extension reaction, two reaction tanks, i.e., the primer extension reaction tank and the PPi reaction tank are present in a single reaction vessel. Requirement of temperature regulation in the treatment of the primer extension reaction, in general, is as described above. More specifically, for example, when a PCR method is employed, the temperature must be raised/lowered in the range of approximately from 50° C. to 90° C., while the temperature must be kept constant at around 65° C. when a LAMP (Loop-Mediated Isothermal Amplification) method or the like is employed. Such a temperature regulation function may be provided to the reaction vessel itself, or may be provided to the detection device, however, in either case, such a temperature regulation function allows the solution in the primer extension reaction tank to stand in a high temperature condition temporarily. In such a case, if H⁺-PPase that is unstable to heat is used, any means for strict temperature regulation must be provided so that the effect of such a high temperature condition is not exerted on the PPi reaction tank when the primer extension reaction tank is allowed to stand in such a high temperature condition.

However, because the mycobacterial H⁺-PPase is very thermostable as described above, such strict temperature regulation is not required. Particularly, the mycobacterial H⁺-PPase retains 60% or more enzyme activity even after being exposed to the condition of 60° C. for 30 minutes. Such thermotolerance is greatly advantageous particularly in cases of use in a LAMP method. In other words, the LAMP method is carried out while keeping the temperature condition of around 65° C. as described above, there is no possibility of complete inactivation in case of the mycobacterial H⁺-PPase even though the PPi reaction tank is then allowed to stand in a condition of 65° C.

Moreover, advantages of seldom inhibition of the enzyme activity of the mycobacterial H⁺-PPase due to a Tris-based buffer are as described above.

With respect to thermostability of H⁺-PPase, H⁺-PPase of thermophilic bacteria such as Thermotoga maritime and Pyrobaculum aerophilum has been known to be also thermotolerant in addition to the mycobacterial H⁺-PPase (see, FEBS Letters 496 (2001) 6-11, and FEBS Letters 460 (1999) 505-512). More specifically, it is reported that optimal temperature of Thermotoga maritime H⁺-PPase is 70° C., and optimal temperature of Pyrobaculum aerophilum H⁺-PPase is 90° C. Therefore, merely taking into account of thermostability alone, greater effect than the cases in which mycobacterial H⁺-PPase is used shall be achieved when H⁺-PPase derived from these thermophilic bacteria is used in the Embodiments 1 to 5 described above.

Meanwhile, the present inventor established in Escherichia coli an extremely efficient expression system of mycobacterial H⁺-PPase which is difficult to produce on a large scale because of low proliferation velocity of mycobacteria, therefore, a large amount of mycobacterial H⁺-PPase can be readily prepared. To the contrary, no expression system of the aforementioned two thermophilic bacterial H⁺-PPase in Escherichia coli has been established, therefore, it is impossible at present to rapidly prepare H⁺-PPase derived from these thermophilic bacteria in large quantities. Therefore, taking into account of aspects of industrial applicability, use of the mycobacterial H⁺-PPase is greatly advantageous.

The method of quantitative determination of PPi and the method of detecting a primer extension reaction, and the kit and device in which the method is performed according to the present invention requires just fewer kinds of enzymes through using mycobacterial H⁺-PPase, in comparison with techniques relating to conventional method of quantitative determination of PPi and method of detecting a primer extension reaction, and also, problems involving instability to heat can be concomitantly overcome. In addition, noteworthy characteristic that its enzyme activity is scarcely inhibited by a Tris-based buffer is also exhibited. Accordingly, the method of measuring PPi and detecting a primer extension reaction, and the kit and device in which the method is performed according to the present invention have very excellent characteristics in terms of storage stability and easy handling in comparison with the case in which conventional H⁺-PPase is used.

In particular, the method of detecting a primer extension reaction, and the kit and device for detection according to the present invention are useful in SNP, diagnoses of mutation, examinations of foods on contamination with bacterium, virus or the like, examinations of human bodies on infection with a bacterium, virus or the like, and the like.

1. A method of measuring pyrophosphate which comprises:
step (a) of adding a solution comprising pyrophosphate into a first region among the first region and a second region defined by a membrane that retains mycobacterial H⁺-PPase and that is H⁺ impermeable such that the solution is brought into contact with said membrane; and
step (b) of measuring H⁺ concentration of either one of said first region or said second region following the step (a),
wherein the active site that hydrolyzes pyrophosphate of said mycobacterial H⁺-pyrophosphatase is exposed to said first region.

2. The method of measuring pyrophosphate according to claim 1 wherein said solution comprises a Tris buffer.

3. The method of measuring pyrophosphate according to claim 1 wherein the H⁺ concentration of either one of said first region or said second region is optically measured in said step (b).

4. The method of measuring pyrophosphate according to claim 3 wherein a pH sensitive dye or a membrane potential sensitive dye is added to at least one of said first region or said second region, and the H⁺ concentration is measured by analysis of an optical characteristic of said pH sensitive dye or membrane potential sensitive dye in said step (b).

5. The method of measuring pyrophosphate according to claim 4 wherein said pH sensitive dye or membrane potential sensitive dye is at least one of the group consisting of pyranin, fluorescein isothiocyanate-dextran, acridine orange, quinacrine and oxonol V.

6. The method of measuring pyrophosphate according to claim 1 wherein the H⁺ concentration of either one of said first region or said second region is electrically measured in said step (b).

7. The method of measuring pyrophosphate according to claim 1 wherein said mycobacterium is Streptomyces coelicolor.

8. A device for measuring pyrophosphate which comprises:
a container,
a H⁺ impermeable membrane that defines said container into an internal region and an external region;
a reference electrode provided so that it is brought into contact with a solution reserved in said external region or internal region; and
a H⁺ sensitive electrode provided so that it is brought into contact with a solution reserved in said internal region,
wherein said membrane has an active site that hydrolyzes pyrophosphate of mycobacterial H⁺-pyrophosphatase such that it is exposed to said external region.

9. The device for measuring pyrophosphate according to claim 8 wherein said solution comprises a Tris buffer.

10. The device for measuring pyrophosphate according to claim 8 wherein said mycobacterium is Streptomyces coelicolor.

11. A method of detecting a primer extension reaction using the method of measuring pyrophosphate according to claim 1,
said method comprising, prior to said step (a), a step (c) of preparing a reaction solution which comprises a subject nucleic acid and a primer having a base sequence that complementarily binds to said subject nucleic acid, and produces pyrophosphate when the extension reaction of said primer occurs,
wherein in said step (a), said reaction solution that will contain pyrophosphate produced upon occurrence of the extension reaction of the primer in said step (c) is added to said first region such that it is brought into contact with said membrane, and the presence of a particular base sequence or base type in said subject nucleic acid is discriminated by measuring pyrophosphate in said reaction solution.

12. The method of detecting a primer extension reaction according to claim 11 wherein the H⁺ concentration is optically measured in said step (b).

13. The method of detecting a primer extension reaction according to claim 11 wherein a pH sensitive dye or a membrane potential sensitive dye is added to at least one of said first region or said second region, and the H⁺ concentration is measured by analysis of an optical characteristic of said pH sensitive dye or membrane potential sensitive dye in said step (b).

14. The method of detecting a primer extension reaction according to claim 13 wherein said pH sensitive dye or membrane potential sensitive dye is at least one of the group consisting of pyranin, fluorescein isothiocyanate-dextran, acridine orange, quinacrine and oxonol V.

15. The method of detecting a primer extension reaction according to claim 11 wherein the H⁺ concentration of at least one of said first region or said second region is electrically measured in said step (b).

16. A device for detecting a primer extension reaction comprising:
a sample inlet for injecting a sample,
a primer extension reaction tank for carrying out a treatment of a primer extension reaction,
a pyrophosphate reaction tank for allowing a reaction for the measurement of pyrophosphate, and
a flow path for connecting said primer extension reaction tank and the pyrophosphate reaction tank,
wherein said primer extension reaction tank reserves a reaction solution which is a reaction solution containing a nucleic acid, and a primer having a base sequence comprising a complementary binding region that complementarily binds to the nucleic acid, and which produces pyrophosphate when an extension reaction of said primer occurs,
said pyrophosphate reaction tank has a detection device for detecting a signal generated within the tank, by the reference electrode and H⁺ sensitive electrode, and
wherein a solution containing pyrophosphate produced by the primer extension reaction is added such that it is brought into contact with said first region among the first region and the second region defined by a membrane that retains mycobacterial H⁺-pyrophosphatase and that is H⁺ impermeable, and thereafter, H⁺ concentration of either one of said first region or said second region is measured.

17. The device for detecting a primer extension reaction according to claim 16 further comprising a temperature regulation means for regulating the temperature of said primer extension reaction tank.

18. The device for detecting a primer extension reaction according to claim 16 further comprising an analysis means for analyzing the measurement result in the aforementioned detection device.
